(19) United States
(12) Patent Application Publication
(10) Pub. No.: US 2018/0338510 A1
(43) Pub. Date: Nov. 29, 2018

(54) WATER AND OTHER LIQUIDS CONTAINING HYDROGEN AND/OR NOBLE GASES

(71) Applicant: Perricone Hydrogen Water Company, LLC, Meriden, CT (US)

(72) Inventor: Nicholas V. Perricone, Madison, CT (US)

(73) Assignee: Perricone Hydrogen Water Company, LLC, Meriden, CT (US)

(21) Appl. No.: 15/834,262

(22) Filed: Dec. 7, 2017

Related U.S. Application Data
(60) Provisional application No. 62/510,102, filed on May 23, 2017.

(51) Int. Cl. A23L 2/54; A61K 33/00; A61K 9/00; B65D 85/72

(52) U.S. Cl. CPC A23L 2/54; A61K 33/00; A23V 2002/00; B65D 85/72; A61K 9/0095

(57) ABSTRACT

Embodiments described herein generally relate to liquids such as water containing hydrogen and a noble gas such as xenon. Such liquids may be useful, for example, for increasing a subject's physical energy levels, for improvement in athletic performance, or the like. For example, the liquid may be in the form of an energy drink. The liquids (e.g., aqueous solutions) and articles described herein may be orally administered to a subject, e.g., drunk by a subject. Some embodiments relate to liquids disposed within a container (e.g., a sealed container), such that the liquid comprises hydrogen gas and a noble gas dissolved or otherwise contained (e.g., infused) in the liquid. In certain embodiments, the hydrogen gas and/or noble gas may be present in the liquid at a particular concentration (e.g., a physiologically relevant concentration). Advantageously, the compositions, articles, and methods described herein may provide liquids containing hydrogen gas and a noble gas that are shelf-stable (e.g., maintain a relatively stable concentration of hydrogen gas and/or noble gas(es)) for relatively long periods of time (e.g., at least 7 days).

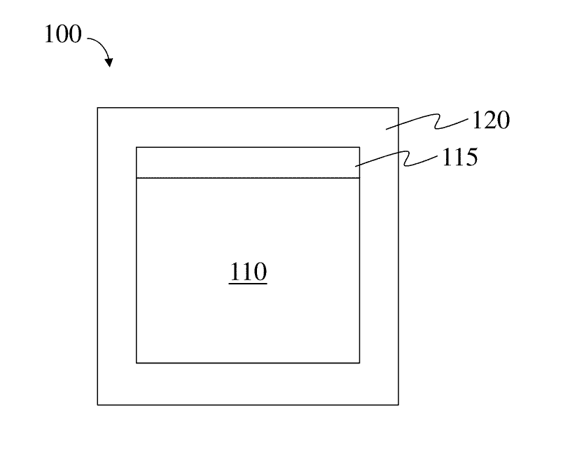

### RELATED APPLICATIONS

This application claims the benefit of U.S. Provisional Patent Application Ser. No. 62/510,102, filed May 23, 2017, entitled “Water and Other Liquids Containing Hydrogen and/or Noble Gases,” by Perricone, which is incorporated herein by reference in its entirety.

### FIELD OF THE INVENTION

The present invention generally relates to liquids containing hydrogen and a noble gas such as xenon, and related methods.

### BACKGROUND

H₂ has been shown to have positive effects on animal and human physiology and disease states. H₂ can be administered to a subject in the form of, for example, a gas, an infusion, a topical solution, or through the drinking of H₂-enriched water. Production of hydrogen-rich water has been accomplished by several methods, ranging from large-scale, but less self-stable, manufacturing techniques to small-volume single use devices for locally generating hydrogen gas.

### SUMMARY OF THE INVENTION

The present invention provides liquids containing hydrogen and a noble gas such as xenon, and related methods. The subject matter of the present invention involves, in some cases, interrelated products, alternative solutions to a particular problem, and/or a plurality of different uses of one or more systems and/or articles.

In one aspect, the present invention is generally directed to an article. In one set of embodiments, the article comprises an aqueous solution contained within a container. In some cases, the aqueous solution comprises dissolved hydrogen gas in a concentration greater than 1 ppm ppm and dissolved xenon gas in a concentration greater than 1 ppm.

The article, according to another set of embodiments, comprises a sealed container containing an aqueous solution and a headspace. In some instances, the aqueous solution comprises dissolved hydrogen gas and dissolved xenon gas. According to certain embodiments, hydrogen gas is present in the headspace in a concentration greater than or equal to 0.00001 vol %, 0.0001 vol %, 0.001 vol %, 0.01 vol %, 0.1 vol %, or 1 vol % versus the total volume of the headspace.

In another set of embodiments, the article comprises a sealed container containing an aqueous solution and a headspace, where the aqueous solution comprises dissolved hydrogen gas and dissolved xenon gas. In some embodiments, xenon gas is present in the headspace in a concentration greater than or equal to 0.00001 vol %, 0.0001 vol %, 0.001 vol %, 0.01 vol %, 0.1 vol %, or 1 vol % versus the total volume of the headspace.

The present invention, in another aspect, is generally directed to a composition. According to one set of embodiments, the composition comprises an aqueous solution, where the aqueous solution comprises dissolved hydrogen gas in an amount of greater than 1 ppm and dissolved xenon gas in an amount of greater than 1 ppm. In some cases, the aqueous solution may be non-toxic.

In accordance with another set of embodiments, the composition comprises an aqueous solution consisting essentially of water, dissolved hydrogen gas in a concentration greater than 1 ppm, and a dissolved noble gas in a concentration greater than 1 ppm.

The present invention, in yet another aspect, is generally directed to a method. In one set of embodiments, the method includes an act of orally administering an aqueous solution to a subject. In some embodiments, the aqueous solution comprises dissolved hydrogen gas in a concentration greater than 1 ppm and dissolved xenon gas in a concentration greater than 1 ppm.

According to another set of embodiments, the method includes drinking an aqueous solution. In certain embodiments, the aqueous solution comprises dissolved hydrogen gas in a concentration greater than 1 ppm, and dissolved xenon gas in a concentration greater than 1 ppm.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

The FIGURE is a schematic drawing illustrating an article comprising a liquid disposed within a container, according to one set of embodiments.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every FIGURE, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

Embodiments described herein generally relate to liquids such as water containing hydrogen and a noble gas such as xenon. Such liquids may be useful, for example, for increasing a subject's physical energy levels, for improvement in athletic performance, or the like. For example, the liquid may be in the form of an energy drink. The liquids (e.g., aqueous solutions) and articles described herein may be orally administered to a subject, e.g., drunk by a subject. Some embodiments relate to liquids disposed within a container (e.g., a sealed container), such that the liquid comprises hydrogen gas and a noble gas dissolved or otherwise contained (e.g., infused) in the liquid. In certain embodiments, the hydrogen gas and/or noble gas may be present in the liquid at a particular concentration (e.g., a physiologically relevant concentration). Advantageously, the compositions, articles, and methods described herein may provide liquids containing hydrogen gas and a noble gas that are shelf-stable (e.g., maintain a relatively stable concentration of hydrogen gas and/or noble gas(es)) for relatively long periods of time (e.g., at least 7 days).

In one aspect, generally, the composition comprises a liquid comprising hydrogen gas and a noble gas. In some embodiments, the liquid comprises hydrogen gas and a noble gas dissolved in water. The liquid may be water, optionally with a variety of additives, such as sugar, electrolytes, caffeine, salt, flavoring, vitamins, herbs, amino acids, tea extracts, seed extracts, fruit extracts. The liquid may be any of a variety of drinkable liquids, such as a fruit juice, coffee, tea, a sports drink, an energy drink, soda pop, milk, an alcoholic drink, or the like.

The hydrogen gas may be present in the liquid in a particular amount (e.g., an amount that would have a significant effect on the body of a subject). The hydrogen gas may be dissolved and/or suspended in the liquid. In some embodiments, the hydrogen gas is present in the liquid in an amount of greater than or equal to 0.1 ppm, greater than or equal to 0.2 ppm, greater than or equal to 0.3 ppm, greater than or equal to 0.4 ppm, greater than or equal to 0.5 ppm, greater than or equal to 0.6 ppm, greater than or equal to 0.7 ppm, greater than or equal to 0.8 ppm, greater than or equal to 0.9 ppm, greater than or equal to 1 ppm, greater than or equal to 1.1 ppm, greater than or equal to 1.2 ppm, greater than or equal to 1.25 ppm, greater than or equal to 1.3 ppm, greater than or equal to 1.4 ppm, greater than or equal to 1.5 ppm, greater than or equal to 1.6 ppm, greater than or equal to 1.7 ppm, greater than or equal to 1.75 ppm, greater than or equal to 1.8 ppm, greater than or equal to 1.9 ppm, greater than or equal to 2 ppm, greater than or equal to 2.25 ppm, greater than or equal to 2.5 ppm, greater than or equal to 2.75 ppm, greater than or equal to 3 ppm, greater than or equal to 3.25 ppm, greater than or equal to 3.5 ppm, greater than or equal to 3.75 ppm, greater than or equal to 4 ppm, greater than or equal to 4.25 ppm, greater than or equal to 4.5 ppm, or greater than or equal to 4.75 ppm.

In certain embodiments, the hydrogen gas is present in the liquid in an amount of less than or equal to 5 ppm, less than or equal to 4.75 ppm, less than or equal to 4.5 ppm, less than or equal to 4.25 ppm, less than or equal to 4 ppm, less than or equal to 3.75 ppm, less than or equal to 3.5 ppm, less than or equal to 3.25 ppm, less than or equal to 3 ppm, less than or equal to 2.75 ppm, less than or equal to 2.5 ppm, less than or equal to 2.25 ppm, less than or equal to 2 ppm, less than or equal to 1.9 ppm, less than or equal to 1.8 ppm, less than or equal to 1.75 ppm, less than or equal to 1.7 ppm, less than or equal to 1.6 ppm, less than or equal to 1.5 ppm, less than or equal to 1.4 ppm, less than or equal to 1.3 ppm, less than or equal to 1.25 ppm, less than or equal to 1.2 ppm, less than or equal to 1.1 ppm, less than or equal to 1 ppm, less than or equal to 0.9 ppm, less than or equal to 0.8 ppm, less than or equal to 0.7 ppm, less than or equal to 0.6 ppm, less than or equal to 0.5 ppm, less than or equal to 0.4 ppm, less than or equal to 0.3 ppm, or less than or equal to 0.2 ppm. Combinations of the above-referenced ranges are also possible (e.g., greater than or equal to 0.1 ppm and less than or equal to 5 ppm, greater than or equal to 1.5 ppm and less than or equal to 2 ppm). Other ranges are also possible.

Non-limiting examples of suitable noble gases that may be present in the liquid include helium, neon, argon, krypton, xenon, and radon. In a particular set of embodiments, the noble gas is xenon gas. In some cases, two or more noble gases may be present, e.g., each independently at the concentrations below. The noble gas may be substantially dissolved and/or suspended in the liquid. For example, the mole fraction solubility of xenon in water at 25° C. and 1 atm is generally 7.890×10⁻⁵. In some embodiments, the amount of noble gas dissolved in the liquid is greater than the amount of noble gas that would be dissolved in the liquid at the mole fraction solubility of the noble gas in water determined at 25° C. and 1 atm. For example, the liquid may be under a pressure greater than 1 atm and/or a temperature greater than 25° C., e.g., as discussed below, which may facilitate greater amounts.

The noble gas may be present in the liquid in a particular amount. For example, in some embodiments, the noble gas is present in an amount of greater than or equal to 1 ppm, greater than or equal to 1.25 ppm, greater than or equal to 1.5 ppm, greater than or equal to 1.75 ppm, greater than or equal to 2 ppm, greater than or equal to 2.25 ppm, greater than or equal to 2.5 ppm, greater than or equal to 2.75 ppm, greater than or equal to 3 ppm, greater than or equal to 3.25 ppm, greater than or equal to 3.5 ppm, greater than or equal to 3.75 ppm, greater than or equal to 4 ppm, greater than or equal to 4.25 ppm, greater than or equal to 4.5 ppm, greater than or equal to 4.75 ppm, greater than or equal to 5 ppm, greater than or equal to 5.5 ppm, greater than or equal to 6 ppm, greater than or equal to 6.5 ppm, greater than or equal to 7 ppm, greater than or equal to 7.5 ppm, greater than or equal to 8 ppm, greater than or equal to 8.5 ppm, greater than or equal to 9 ppm, greater than or equal to 9.5 ppm, greater than or equal to 10 ppm, greater than or equal to 11 ppm, greater than or equal to 12 ppm, greater than or equal to 13 ppm, greater than or equal to 14 ppm, greater than or equal to 15 ppm, greater than or equal to 16 ppm, greater than or equal to 17 ppm, greater than or equal to 18 ppm, or greater than or equal to 19 ppm.

In certain embodiments, the noble gas is present in the liquid in an amount of less than or equal to 20 ppm, less than or equal to 19 ppm, less than or equal to 18 ppm, less than or equal to 17 ppm, less than or equal to 16 ppm, less than or equal to 15 ppm, less than or equal to 14 ppm, less than or equal to 13 ppm, less than or equal to 12 ppm, less than or equal to 11 ppm, less than or equal to 10 ppm, less than or equal to 9.5 ppm, less than or equal to 9 ppm, less than or equal to 8.5 ppm, less than or equal to 8 ppm, less than or equal to 7.5 ppm, less than or equal to 7 ppm, less than or equal to 6.5 ppm, less than or equal to 5 ppm, less than or equal to 4.75 ppm, less than or equal to 4.5 ppm, less than or equal to 4.25 ppm, less than or equal to 4 ppm, less than or equal to 3.75 ppm, less than or equal to 3.5 ppm, less than or equal to 3.25 ppm, less than or equal to 3 ppm, less than or equal to 2.75 ppm, less than or equal to 2.5 ppm, less than or equal to 2.25 ppm, less than or equal to 2 ppm, less than or equal to 1.75 ppm, less than or equal to 1.5 ppm, or less than or equal to 1.25 ppm. Combinations of the above-referenced ranges are also possible (e.g., greater than or equal to 1 ppm and less than or equal to 20 ppm, greater than or equal to 10 ppm and less than or equal to 15 ppm). Other ranges are also possible. The noble gas may include xenon, and/or other gases as discussed herein.

In some cases, the liquid may be present in a container. For example, as illustrated schematically in the FIGURE, article 100 may comprise a liquid (for example, an aqueous solution) 110 disposed in container 120. In certain embodiments, the container may be sealed, e.g., to the external atmosphere. For example, in certain embodiments, the container may be sealed such that the liquid and/or gases (e.g., hydrogen gas, and/or xenon gas) within the container are not able to substantially exit the container. In some cases, the seal may be removable (e.g., such that the liquid may be removed from the container and/or orally administered to a subject, e.g., drunk). For example, in an exemplary embodiment, the container is a can and the can may be unsealed by breaking the seal of the can (e.g., via a pull-tab, push-tab, or stay-tab associated with the seal). In another exemplary embodiment, the container is a bottle or pouch, and the container may be unsealed by removing a cap associated with an opening of the container. Upon unsealing of the container, the liquid may be ingested (e.g., drunk) by the subject.

Non-limiting examples of suitable types of containers include cans (e.g., aluminum or tin cans), bottles, jars, pouches, boxes, and bags. Other containers are also possible and those of ordinary skill in the art would be capable of selecting suitable containers based upon the teachings of this specification. The container may also comprise any suitable material. For example, in some embodiments, the container may comprise a material such as metal (e.g., aluminum, tin, iron), metal alloys (e.g., steel), polymer (e.g., polyethylene, polystyrene, polypropylene, polyether ether ketones, polyethylene terephthalate, polyvinylchloride), glass (e.g., borosilicate glass), resin, and combinations thereof. In some cases, the container is able to contain an elevated pressure therein (e.g., a pressure greater than atmospheric pressure).

In addition, in some embodiments, one or more coatings or other materials may be used to facilitate retention of gases within the container, e.g., such materials may be relatively gas-impermeable. A variety of gas-impermeable materials may be readily obtained commercially, and coated onto a surface of the container and/or embedded within the materials forming the container. Non-limiting examples of gas-impermeable materials include polyester, nylon (e.g., MXD6 nylon or nylon 6), ethylene vinyl alcohol (EVA), silicon oxides (SiOₓ), or the like.

Thus, in some embodiments, the liquid is contained within a container at a particular pressure that may be greater than atmospheric pressure. The pressure may be created within the container using any of a variety of gases, including air, nitrogen, carbon dioxide, water vapor, hydrogen gas, one or more noble gases (such as xenon), or the like, as well as combinations of these and/or other suitable gases. Such gases may be at equilibrium with the liquid within the container. In addition, in some cases, one or more of the gases may be present in an amount such that at equilibrium, those gases are dissolved within the liquid at saturation concentrations.

For example, in certain embodiments, the container contains a pressure at least 1 psi (1 psi is about 6894.757 Pa), at least 2 psi, at least 3 psi, at least 5 psi, at least 7 psi, at least 10 psi, at least 12 psi, at least 15 psi, at least 18 psi, at least 20 psi, at least 25 psi, at least 30 psi, at least 35 psi, at least 40 psi, or at least 45 psi greater than atmospheric pressure. In some embodiments, the container contains a pressure of less than or equal to 50 psi, less than or equal to 45 psi, less than or equal to 40 psi, less than or equal to 35 psi, less than or equal to 30 psi, less than or equal to 25 psi, less than or equal to 20 psi, less than or equal to 18 psi, less than or equal to 15 psi, less than or equal to 12 psi, less than or equal to 10 psi, less than or equal to 7 psi, less than or equal to 5 psi, less than or equal to 3 psi, or less than or equal to 2 psi greater than atmospheric pressure. Combinations of the above-referenced ranges are also possible (e.g., at least 1 psi and less than or equal to 50 psi greater than atmospheric pressure). Other ranges are also possible.

In some embodiments, the article comprises a gaseous headspace (e.g., a gaseous headspace present within the container). For example, referring again to the FIGURE, in some cases, article 100 comprises gaseous headspace 115. The article may comprise any suitable amount of headspace within the container. In some embodiments, the headspace occupies greater than or equal to 0.1 vol %, greater than or equal to 0.2 vol %, greater than or equal to 0.25 vol %, greater than or equal to 0.5 vol %, greater than or equal to 0.75 vol %, greater than or equal to 1 vol %, greater than or equal to 1.25 vol %, greater than or equal to 1.5 vol %, greater than or equal to 1.75 vol %, greater than or equal to 2 vol %, greater than or equal to 2.25 vol %, greater than or equal to 2.5 vol %, greater than or equal to 3 vol %, greater than or equal to 3.5 vol %, greater than or equal to 4 vol %, or greater than or equal to 4.5 vol % of the volume contained by the container. In certain embodiments, the headspace occupies less than or equal to 5 vol %, less than or equal to 4.5 vol %, less than or equal to 4 vol %, less than or equal to 3.5 vol %, less than or equal to 3 vol %, less than or equal to 2.5 vol %, less than or equal to 2.25 vol %, less than or equal to 2 vol %, less than or equal to 1.75 vol %, less than or equal to 1.5 vol %, less than or equal to 1.25 vol %, less than or equal to 1 vol %, less than or equal to 0.75 vol %, less than or equal to 0.5 vol %, less than or equal to 0.25 vol %, or less than or equal to 0.2 vol % of the volume contained by the container. Combinations of the above-referenced ranges are also possible (e.g., greater than or equal to 0.1 vol % and less than or equal to 5 vol %). Other ranges are also possible.

In some embodiments, the gaseous headspace comprises hydrogen gas. In certain embodiments, the gaseous headspace comprises a noble gas such as xenon gas. In some cases, both hydrogen gas and the noble gas (e.g., xenon gas) may be present in the headspace. As mentioned, other gases (including other noble gases) may be present as well, e.g., as discussed herein. In some embodiments, when hydrogen gas and/or a noble gas such as xenon gas are present in the headspace, the concentration of hydrogen gas and/or xenon gas greater than the concentration of the gas that would result from the saturation vapor pressure of the gas.

For example, in some embodiments, the headspace comprises greater than or equal to 0.00001 vol %, greater than or equal to 0.00005 vol %, greater than or equal to 0.0001 vol %, greater than or equal to 0.0005 vol %, greater than or equal to 0.001 vol %, greater than or equal to 0.005 vol %, greater than or equal to 0.01 vol %, greater than or equal to 0.05 vol %, greater than or equal to 0.1 vol %, greater than or equal to 0.5 vol %, greater than or equal to 1 vol %, greater than or equal to 2 vol %, greater than or equal to 5 vol %, greater than or equal to 10 vol %, greater than or equal to 20 vol %, greater than or equal to 30 vol %, greater than or equal to 40 vol %, greater than or equal to 50 vol %, or greater than or equal to 60 vol % xenon gas (and/or other noble gases) versus the total volume of the headspace. In certain embodiments, xenon gas (and/or other noble gases) is present in the headspace in an amount less than or equal to 70 vol %, less than or equal to 60 vol %, less than or equal to 50 vol %, less than or equal to 40 vol %, less than or equal to 30 vol %, less than or equal to 20 vol %, less than or equal to 10 vol %, less than or equal to 5 vol %, less than or equal to 2 vol %, less than or equal to 1 vol %, less than or equal to 0.5 vol %, less than or equal to 0.1 vol %, less than or equal to 0.05 vol %, less than or equal to 0.01 vol %, less than or equal to 0.005 vol %, less than or equal to 0.001 vol %, less than or equal to 0.0005 vol %, less than or equal to 0.0001 vol %, or less than or equal to 0.00005 vol % versus the total volume of the headspace. Combinations of the above-referenced ranges are also possible (e.g., greater than or equal to 0.00001 vol % and less than or equal to 10 vol %). Other ranges are also possible.

In certain embodiments, the headspace comprises greater than or equal to 0.00001 vol %, greater than or equal to 0.00005 vol %, greater than or equal to 0.0001 vol %, greater than or equal to 0.0005 vol %, greater than or equal to 0.001 vol %, greater than or equal to 0.005 vol %, greater than or equal to 0.01 vol %, greater than or equal to 0.05 vol %, greater than or equal to 0.1 vol %, greater than or equal to 0.5 vol %, greater than or equal to 1 vol %, greater than or equal to 2 vol %, or greater than or equal to 5 vol % hydrogen gas versus the total volume of the headspace. In certain embodiments, hydrogen gas is present in the headspace in an amount less than or equal to 10 vol %, less than or equal to 5 vol %, less than or equal to 2 vol %, less than or equal to 1 vol %, less than or equal to 0.5 vol %, less than or equal to 0.1 vol %, less than or equal to 0.05 vol %, less than or equal to 0.01 vol %, less than or equal to 0.005 vol %, less than or equal to 0.001 vol %, less than or equal to 0.0005 vol %, less than or equal to 0.0001 vol %, or less than or equal to 0.00005 vol % versus the total volume of the headspace. Combinations of the above-referenced ranges are also possible (e.g., greater than or equal to 0.00001 vol % and less than or equal to 10 vol %). Other ranges are also possible.

In one set of embodiments, the liquid within the sealed container fills greater than or equal to 50 vol %, greater than or equal to 75 vol %, greater than or equal to 80 vol %, greater than or equal to 85 vol %, greater than or equal to 90 vol %, greater than or equal to 92 vol %, greater than or equal to 95 vol %, greater than or equal to 98 vol %, greater than or equal to 99 vol %, greater than or equal to 99.5 vol %, or greater than or equal to 99.9 vol % of the volume of the sealed container. In some cases, the volume of the liquid may be less than or equal to 99.99 vol %, less than or equal to 99.9 vol %, less than or equal to 99.5 vol %, less than or equal to 99 vol %, less than or equal to 98 vol %, less than or equal to 95 vol %, less than or equal to 92 vol %, less than or equal to 90 vol %, less than or equal to 85 vol %, less than or equal to 80 vol %, or less than or equal to 75 vol % of the volume of the sealed container. Combinations of the above-referenced ranges are also possible (e.g., greater than or equal to 50 vol % and less than or equal to 99.99 vol %).

In some embodiments, the articles (e.g., a sealed container) described herein are configured to have a relatively long shelf life with respect to the gases contained therein. In certain embodiments, the hydrogen gas and noble gas (e.g., xenon gas) does not substantially leak from the sealed container for at least 7 days, or longer (e.g., 14 days, 28 days, 56 days, etc.). For example, in some embodiments, greater than or equal to 50 vol %, greater than or equal to 75 vol %, greater than or equal to 80 vol %, greater than or equal to 85 vol %, greater than or equal to 90 vol %, greater than or equal to 92 vol %, greater than or equal to 95 vol %, greater than or equal to 98 vol %, greater than or equal to 99 vol %, greater than or equal to 99.5 vol %, or greater than or equal to 99.9 vol % of the hydrogen gas and/or xenon gas (and/or other noble gases) is present in the sealed container and/or in the headspace 7 days after sealing of the container (including the liquid comprising the hydrogen gas and the noble gas). In certain embodiments, less than or equal to 99.99 vol %, less than or equal to 99.9 vol %, less than or equal to 99.5 vol %, less than or equal to 99 vol %, less than or equal to 98 vol %, less than or equal to 95 vol %, less than or equal to 92 vol %, less than or equal to 90 vol %, less than or equal to 85 vol %, less than or equal to 80 vol %, or less than or equal to 75 vol % of the hydrogen gas and/or xenon gas (and/or other noble gases) is present in the sealed container and/or in the headspace 7 days after sealing of the container (including within the liquid comprising the hydrogen gas and the noble gas). Combinations of the above-referenced ranges are also possible (e.g., greater than or equal to 50 vol % and less than or equal to 99.99 vol %). Other ranges are also possible.

In some embodiments, one or more additives may be present. Non-limiting examples of additives include sugar, electrolytes, caffeine, salt, flavoring, vitamins, herbs, amino acids, tea extracts, seed extracts, fruit extracts, and combinations thereof. The one or more additives may be present in any suitable amount. For example, in some embodiments, the additive is present in the liquid in an amount of greater than or equal to 0.1 vol %, greater than or equal to 0.2 vol %, greater than or equal to 0.25 vol %, greater than or equal to 0.5 vol %, greater than or equal to 0.75 vol %, greater than or equal to 1 vol %, greater than or equal to 1.25 vol %, greater than or equal to 1.5 vol %, greater than or equal to 1.75 vol %, greater than or equal to 2 vol %, greater than or equal to 2.25 vol %, greater than or equal to 2.5 vol %, greater than or equal to 3 vol %, greater than or equal to 3.5 vol %, greater than or equal to 4 vol %, or greater than or equal to 4.5 vol % versus the total volume of the liquid.

In certain embodiments, the additive is present in the liquid solution in an amount less than or equal to 5 vol %, less than or equal to 4.5 vol %, less than or equal to 4 vol %, less than or equal to 3.5 vol %, less than or equal to 3 vol %, less than or equal to 2.5 vol %, less than or equal to 2.25 vol %, less than or equal to 2 vol %, less than or equal to 1.75 vol %, less than or equal to 1.5 vol %, less than or equal to 1.25 vol %, less than or equal to 1 vol %, less than or equal to 0.75 vol %, less than or equal to 0.5 vol %, less than or equal to 0.25 vol %, or less than or equal to 0.2 vol % versus the total volume of the liquid. Combinations of the above-referenced ranges are also possible (e.g., greater than or equal to 0.1 vol % and less than or equal to 5 vol %). Other ranges are also possible.

As mentioned, the liquid may be any of a variety of drinkable liquids in various embodiments, such as water, a fruit juice, coffee, tea, a sports drink, an energy drink, soda pop, milk, an alcoholic drink, etc.

In some embodiments, the liquid (e.g., the aqueous solution) comprises hydrogen gas, a noble gas (e.g., xenon gas), and one or more additives, in the ranges listed above with the remainder of the liquid being water. In certain embodiments, water is present in the liquid in an amount of greater than or equal to 90 wt %, greater than or equal to 91 wt %, greater than or equal to 92 wt %, greater than or equal to 93 wt %, greater than or equal to 94 wt %, greater than or equal to 95 wt %, greater than or equal to 96 wt %, greater than or equal to 97 wt %, greater than or equal to 98 wt %, greater than or equal to 99 wt %, greater than or equal to 99.5 wt %, or greater than or equal to 99.9 wt % versus the total liquid weight. In some embodiments, the liquid comprises less than or equal to 99.99 wt %, less than or equal to 99.9 wt %, less than or equal to 99.5 wt %, less than or equal to 99 wt %, less than or equal to 98 wt %, less than or equal to 97 wt %, less than or equal to 96 wt %, less than or equal to 95 wt %, less than or equal to 94 wt %, less than or equal to 93 wt %, less than or equal to 92 wt %, or less than or equal to 91 wt % water versus the total liquid weight. Combinations of the above-referenced ranges are also possible (e.g., greater than or equal to 90 wt % and less than or equal to 99.99 wt %, greater than or equal to 95 wt % and less than or equal to 99.99 wt %, greater than or equal to 98 wt % and less than or equal to 99.99 wt %). Other ranges are also possible.

In certain embodiments, water is present in the liquid in an amount of greater than or equal to 90 vol %, greater than or equal to 91 vol %, greater than or equal to 92 vol %, greater than or equal to 93 vol %, greater than or equal to 94 vol %, greater than or equal to 95 vol %, greater than or equal to 96 vol %, greater than or equal to 97 vol %, greater than or equal to 98 vol %, greater than or equal to 99 vol %, greater than or equal to 99.5 vol %, or greater than or equal to 99.9 vol % versus the total volume of the liquid. In some embodiments, the liquid comprises less than or equal to 99.99 vol %, less than or equal to 99.9 vol %, less than or equal to 99.5 vol %, less than or equal to 99 vol %, less than or equal to 98 vol %, less than or equal to 97 vol %, less than or equal to 96 vol %, less than or equal to 95 vol %, less than or equal to 94 vol %, less than or equal to 93 vol %, less than or equal to 92 vol %, or less than or equal to 91 vol % versus the total volume of the liquid. Combinations of the above-referenced ranges are also possible (e.g., greater than or equal to 90 vol % and less than or equal to 99.99 vol %, greater than or equal to 95 vol % and less than or equal to 99.99 vol %, greater than or equal to 98 vol % and less than or equal to 99.99 vol %). Other ranges are also possible.

In some embodiments, the composition may be used to increase the energy levels and/or overall feeling or well-being of a subject. For example, the composition may reduce oxidative stress and/or reduce muscle fatigue (e.g., after exercise and/or athletic activity). In certain embodiments, the composition may improve a subject's overall well-being including, for example, a feeling of increased energy levels, hastened recovery after exercise, improved memory, increased strength, and/or reduced tiredness. In some cases, the composition may be particularly delectable to the subject. In other embodiments, the composition may provide performance enhancement to a subject while, for example, exercising and/or performing athletic activities. See also a U.S. patent application filed on even date herewith, entitled “Systems and Methods for Treatments Using Hydrogen and/or Noble Gases,” by Perricone, incorporated herein by reference in its entirety.

In certain embodiments, the aqueous solution may be orally administered to a subject, e.g., drunk by a subject. In some such embodiments, the aqueous solution may comprise hydrogen gas in an amount of greater than or equal to greater than or equal to 0.1 ppm and less than or equal to 5 ppm and xenon gas (and/or other noble gases) in an amount of greater than or equal to 1 ppm and less than or equal to 20 ppm.

In certain embodiments, the articles, compositions, and aqueous solutions described herein are substantially non-toxic. The term “non-toxic” refers to a substance that does not comprise a toxic compound. The term “toxic” refers to a substance showing detrimental, deleterious, harmful, or otherwise negative effects on a subject, tissue, or cell when or after administering the substance to the subject or contacting the tissue or cell with the substance, compared to the subject, tissue, or cell prior to administering the substance to the subject or contacting the tissue or cell with the substance. In certain embodiments, the effect is death or destruction of the subject, tissue, or cell. In certain embodiments, the effect is a detrimental effect on the metabolism of the subject, tissue, or cell. In certain embodiments, a toxic substance is a substance that has a median lethal dose (LD50) of not more than 500 milligrams per kilogram of body weight when administered orally to an albino rat weighing between 200 and 300 grams, inclusive. In certain embodiments, a toxic substance is a substance that has an LD50 of not more than 1,000 milligrams per kilogram of body weight when administered by continuous contact for 24 hours (or less if death occurs within 24 hours) with the bare skin of an albino rabbit weighing between two and three kilograms, inclusive. In certain embodiments, a toxic substance is a substance that has an LC50 in air of not more than 2,000 parts per million by volume of gas or vapor, or not more than 20 milligrams per liter of mist, fume, or dust, when administered by continuous inhalation for one hour (or less if death occurs within one hour) to an albino rat weighing between 200 and 300 grams, inclusive.

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon drinking the liquid, or otherwise being orally administered the composition described herein.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

A U.S. patent application filed on even date herewith, entitled “Systems and Methods for Treatments Using Hydrogen and/or Noble Gases,” by Perricone, is incorporated herein by reference in its entirety.

### EXAMPLES

The following examples illustrate embodiments of certain aspects of the invention. It should be understood that the methods and/or materials described herein may be modified and/or scaled, as known to those of ordinary skill in the art.

### Prophetic Example 1

A sealed container contains a liquid comprising water, hydrogen gas, and xenon gas. The hydrogen gas is present in an amount of greater than or equal to 1.5 ppm and less than or equal to 2 ppm. The xenon gas is present in an amount of greater than 10 ppm and less than 15 ppm. A subject unseals and drink the liquid to enjoy the benefits of the liquid, or just to enjoy drinking the liquid itself.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. An article, comprising:
an aqueous solution contained within a container, wherein the aqueous solution comprises dissolved hydrogen gas in a concentration greater than 1 ppm and a dissolved noble gas in a concentration greater than 1 ppm.

2. The article of claim 1, wherein the container is a can, bottle, jar, pouch, box, or bag.

3. The article of claim 1, wherein the container is sealed.

4. The article of claim 1, wherein the container contains a pressure at least 1 psi greater than atmospheric pressure.

5. The article of claim 1, wherein the container further contains a gaseous headspace.

6. The article of claim 5, wherein the gaseous headspace comprises at least 0.00001 vol % H₂.

7. The article of claim 1, wherein the aqueous solution is at least 90 wt % water.

8. The article of claim 1, wherein the aqueous solution is at least 95 wt % water.

9. The article of claim 1, wherein the aqueous solution is at least 98 wt % water.

10. The article of claim 1, wherein the aqueous solution is non-toxic.

11. An article, comprising:
a sealed container containing an aqueous solution and a headspace, wherein the aqueous solution comprises dissolved hydrogen gas and dissolved xenon gas, wherein hydrogen gas is present in the headspace in a concentration greater than or equal to 0.00001 vol % versus the total volume of the headspace.

12-15. (canceled)

16. A method, comprising:
drinking an aqueous solution, wherein the aqueous solution comprises dissolved hydrogen gas in a concentration greater than 1 ppm, and a dissolved noble gas in a concentration greater than 1 ppm.

17. An article as in claim 1, wherein the aqueous solution comprises one or more additives.

18. An article as in claim 17, wherein the one or more additives are selected from the group consisting of sugar, caffeine, salt, flavoring, vitamins, herbs, amino acids, tea extracts, seed extracts, fruit extracts, and combinations thereof.

19. The article of claim 11, wherein the container contains a pressure at least 1 psi greater than atmospheric pressure.

20. The article of claim 11, wherein the container is a can, bottle, jar, pouch, box, or bag.

21. The article of claim 11, wherein the aqueous solution is at least 90 wt % water.

22. The method of claim 16, wherein the aqueous solution is at least 90 wt % water.

23. The method of claim 16, wherein the dissolved noble gas is xenon.

24. The method of claim 16, wherein the aqueous solution is at a pressure at least 1 psi greater than atmospheric pressure.
